(19) United States
(12) Patent Application Publication
(10) Pub. No.: US 2007/0250164 A1
(43) Pub. Date: Oct. 25, 2007

(54) Method for grafting whole superficial articular cartilage

(75) Inventor: Karen Troxel, Warsaw, IN (US)

Correspondence Address: HARNESS, DICKEY & PIERCE, P.L.C., P.O. BOX 828, BLOOMFIELD HILLS, MI, 48303, US

(73) Assignee: Biomet Manufacturing Corp., Warsaw, IN (US)

(21) Appl. No.: 11/409,473

(22) Filed: Apr. 21, 2006

(51) Int. Cl. A61F 2/28

(52) U.S. Cl. 623/14.12; 623/919

(57) ABSTRACT

Methods for grafting whole superficial articular cartilage are provided. A defect site is prepared to receive the whole superficial cartilage graft by removing a region of tissue. A whole superficial cartilage is obtained from a selected articular cartilage site. The whole superficial cartilage graft is attached to the prepared deficit site to sufficiently reduce movement of the whole superficial cartilage.

### FIELD

The present teachings relate to methods and apparatus for repairing cartilage defects.

### BACKGROUND

Articular cartilage enables bones to move smoothly relative to one another. The three zones of cartilage include the superficial zone which is adjacent the articulating surface, the middle zone, and the deep zone which is adjacent the subchondral bone. The superficial zone comprises flattened cells orientated parallel with the cartilage surface. The orientation of the superficial zone cells facilitates the smooth movement of the bones against each other. The cells in the intermediate and deep zones tend to be more spherical and arranged either at random or in columns oriented perpendicular to the articular surface.

Damage to the articular cartilage can be caused by injury, such as tearing, by excessive wear, or by a lifetime of use. Damage to articular cartilage, particularly of the meniscus and load-bearing regions, causes pain and reduced mobility. Damage to the articular cartilage is particularly troubling because damaged articular cartilage does not “heal” completely like other tissues due to the lack of blood and nervous supply in the articular cartilage. Furthermore, when the damage heals naturally, the repair tissue formed is fibrocartilage (generally found in the skin and tendons, for example) which does not have the same biomechanical characteristics as hyaline cartilage (found in the ears and the joints, for example). Accordingly, there is decreased strength and load-bearing abilities of the area.

Medical intervention such as medications, therapy, or surgery can be required to restore proper function to the articular cartilage. Some of the current procedures for treatment of articular cartilage defects include lavage and debridement, abrasion chondroplasty, microfracture techniques, subchondral drilling, transplantation of periosteal or perichondral grafts, and transplantation of osteochondral autografts or allografts, for example. Other techniques include mincing cartilage and placing the minced cartilage into a scaffold and implanting the scaffold into a defect site to promote the ingrowth of new cartilage. These procedures, however, do not lead to successful regeneration of the tissue, do not maximize ingrowth of new cartilage, and/or do not duplicate the mechanical properties and durability of the patient's original healthy articular cartilage. Furthermore, with respect to autografts and allografts, an additional problem is that the graft may not be of an adequate size to cover the defect site. Providing adequately sized grafts, especially in autologous donations, requires that a large graft is excised from a donor site which may in turn compromise the donor site, particularly with full thickness cartilage grafts.

In light of the shortcomings of the current procedures, research has focused on increasing chondrogenesis or the formation of cartilage from chondrocytes—the cartilage producing cells. The focus has included cellular manipulation and techniques to deliver the chondrocytes to cartilage defect sites.

Additional research involving chondroprogenitor cells includes the studies focused on the roles of each zone of cartilage in cartilage regeneration. While not intending to be bound by a particular theory, studies have shown that the chondroprogenic cells in the superficial zone can promote differentiation of other chondroprogenic cells and can also promote chondrogenesis. Nonetheless, the research has been limited to cellular manipulation and development and has not focused on practical ways of implementing and maximizing the superficial zone chondrocytes for surgical methods and repair devices.

Accordingly, there is a need for cartilage implants and surgical methods to exploit the superficial zone to facilitate cartilage repair. There is also a need for simple and effective methods for repairing cartilage defects which do not require the removal of significant and unnecessary portions of donor tissue, such as the subchondral bone. Such methods for cartilage repair are needed to restore patient mobility, alleviate pain, allow for the ingrowth of new healthy cartilage, and simplify surgical efforts.

### SUMMARY

The present teachings provide methods for grafting whole superficial articular cartilage. A defect site is prepared to receive the whole superficial cartilage graft by removing a region of tissue. A whole superficial cartilage is obtained from a selected articular cartilage site. The harvested whole superficial cartilage is attached to the prepared defect site to sufficiently reduce movement of the whole superficial cartilage graft. The whole superficial cartilage graft can also be shaped to correspond with at least a region of the defect site. The whole superficial cartilage graft can also be treated with an agent to enhance growth of the whole superficial cartilage graft. The whole superficial cartilage graft can be attached to the defect site using mechanical securing, adhesive securing, membrane securing, and combinations thereof. The whole superficial cartilage graft can be harvested from an allogeneic donor and preserved for later use. The whole superficial cartilage graft can be harvested from an autologous articular cartilage source.

The whole superficial cartilage graft can include an integral layer of intermediate cartilage. The whole superficial cartilage graft can include slits to allow the graft to expand under tension to fill at least a region of the defect. The slits can be equally distributed across the surface of the whole superficial cartilage graft. The defect site can be in a body region such as the patella, a femoral condyle, a femoral head, or an acetabulum. The whole superficial cartilage graft can have an expansion ratio of from about 1:1 to about 1:10.

A method for repairing knee articular cartilage defects is provided. A defect site located on a condyle surface is prepared by removing a region of tissue. Whole autologous superficial cartilage is harvested from a selected articular cartilage site. The whole autologous superficial cartilage is applied to the prepared defect site. The whole autologous superficial cartilage is attached to the prepared defect site to sufficiently reduce movement of the whole autologous superficial cartilage. The attachment can be mechanical securing, adhesive securing, membrane securing, or combinations thereof. The whole autologous superficial cartilage graft can be sized to fit the defect site. The whole autologous superficial cartilage graft can include an integral layer of intermediate cartilage.

A method for grafting whole superficial articular cartilage is provided. The defect site is prepared to receive a whole superficial cartilage graft by removing a region of tissue. The whole superficial cartilage is removed from a selected articular cartilage site. The whole superficial cartilage is placed in the defect site. The whole superficial cartilage graft is covered with a periosteal flap. The periosteal flap is attached to the prepared defect site. At least a region of the attached periosteal flap can be coated with a fibrin sealant. The attached periosteal flap can be leveled with at least a region of the surrounding healthy tissue.

Further areas of applicability of the present teachings will become apparent from the detailed description provided hereinafter. It should be understood that the detailed description and specific examples, while indicating the preferred embodiment of the teachings, are intended for purposes of illustration only and are not intended to limit the scope of the present teachings.

### BRIEF DESCRIPTION OF THE DRAWINGS

The present teachings will become more fully understood from the detailed description and the accompanying drawings, wherein:

FIG. 1 depicts a top view of a whole superficial cartilage graft according to the present teachings;

FIGS. 2A-2B depict side views of whole superficial cartilage grafts according to the present teachings;

FIGS. 3A-3B depict top views of a whole superficial cartilage graft having expandable surface features according to the present teachings;

FIGS. 4A-4B depict top views of a whole superficial cartilage graft having slits according to the present teachings;

FIG. 5 depicts a side view of whole superficial cartilage grafts having slits according to the present teachings;

FIG. 6 depicts a defect site in the articular cartilage;

FIG. 7 depicts a tool for removing articular cartilage from a non-load bearing region according to the present teachings;

FIG. 8 depicts an exploded view of the articular cartilage removal of FIG. 7;

FIG. 9 depicts an adjustable tool for removing articular cartilage according to the present teachings;

FIGS. 10A-10B depict various tools for meshing the articular cartilage according to the present teachings; and

FIG. 11 depicts a repaired defect site according to the present teachings.

### DETAILED DESCRIPTION

The following description of the various embodiments is merely exemplary in nature and is in no way intended to limit the present teachings, their application, or uses. Although the various examples can be discussed in conjunction with the condyle surfaces of the knee, it is understood that the present teachings can be used in any cartilage containing area.

Referring to FIG. 1, a whole superficial cartilage graft 10 (also referred to as a partial thickness cartilage graft 10) is provided. The grafts are “partial thickness” grafts and include a superficial zone 12 of cartilage and a portion of an intermediate zone 14 of cartilage. The partial thickness graft of various embodiments can also refer to those grafts which consist of only the superficial zone 12 of cartilage. The whole superficial cartilage graft 10 does not include the deep zone of cartilage and does not include the underlying subchondral bone. The advantages of a whole superficial cartilage graft 10 includes removal of less tissue from a donor which expedites healing of the donor region and limits removal to only the damaged cartilage surface tissues from the recipient region. Use of the whole superficial cartilage graft also increases the flexibility of the graft and the extent to which the graft can be manipulated to provide adequate coverage, especially when the graft is smaller than the defect site. Additional advantages include ease of removal and reduction of stress on any underlying bone, as further detailed later herein. The whole superficial cartilage graft 10 is “whole” or the graft is a continuous piece of non-minced cartilage which is harvested to provide maximum coverage to a cartilage defect and minimize the number of grafts needed to cover the defect site. While various embodiments employ a single piece of partial thickness cartilage to effectively cover the defect site, it is understood and examples are provided herein where more than one piece of whole partial thickness cartilage can be used in the defect site.

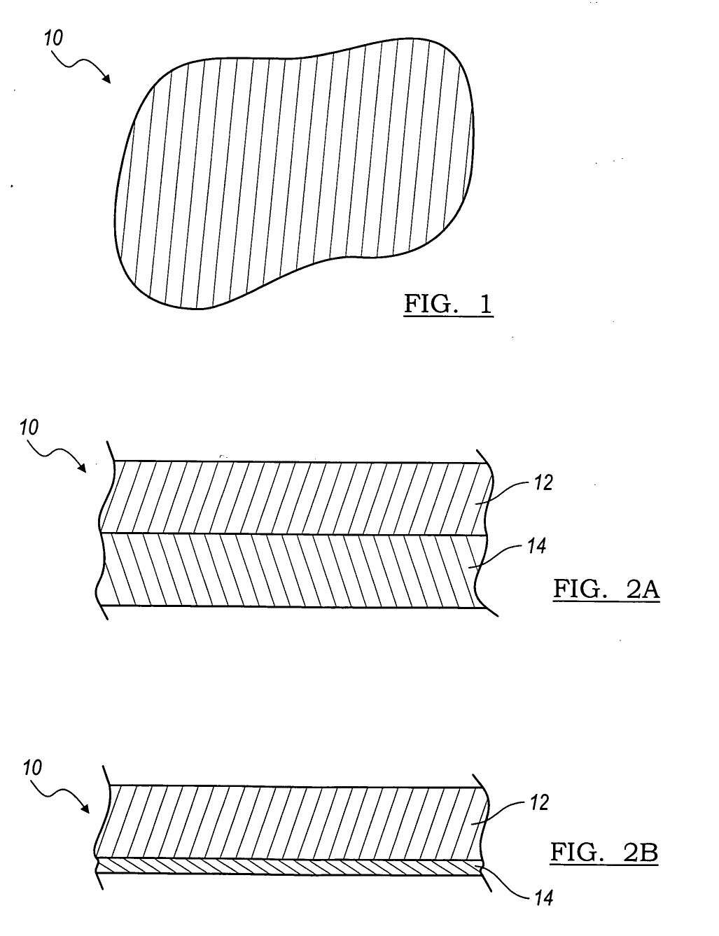

The superficial zone 12 of the whole superficial cartilage graft 10 can include active chondroprogenitor cells. These active chondroprogenitor cells are capable of producing chondrocytes, which will in turn produce cartilage. In various embodiments, the superficial zone 12 can make up about 100% of the whole superficial cartilage graft 10 by volume. As depicted in FIG. 2A, the superficial zone 12 and the intermediate zone 14 can have equal thicknesses in the whole superficial cartilage graft 10. As depicted in FIG. 2B, the superficial zone 12 can be of a greater thickness than the intermediate zone 14. The thickness difference can be achieved when harvesting the whole superficial cartilage graft 10, as discussed later herein. Providing a greater thickness superficial zone 12 increases the amount of chondrocytes provided to the defect site in comparison to a full thickness cartilage graft.

The intermediate zone of cartilage includes cells which are randomly oriented. In various embodiments, the intermediate zone 14 makes up from about 1% to about 50% of embodiments of the whole superficial cartilage graft 10 which include both the superficial zone 12 and the intermediate zone 14. In various other embodiments the intermediate zone 14 makes up at least about 20% of the whole superficial cartilage graft 10. Because of the thinness of the superficial zone 12 of cartilage, it may be desirable to include at least a portion of the intermediate zone 14 to facilitate easy harvesting and/or handling of the partial thickness cartilage implant 10.

Returning to FIG. 1, the whole superficial cartilage graft 10 can be of any suitable shape. Exemplary suitable shapes include discs, squares, rectangles, tear-drop shapes, and free form shapes. The desired shape of the whole superficial cartilage graft 10 can be manipulated by the user, by trimming the whole superficial cartilage graft 10 with surgical scissors, for example. The shape of the whole superficial cartilage graft 10 can be shaped to correspond with or fit into a defect site.

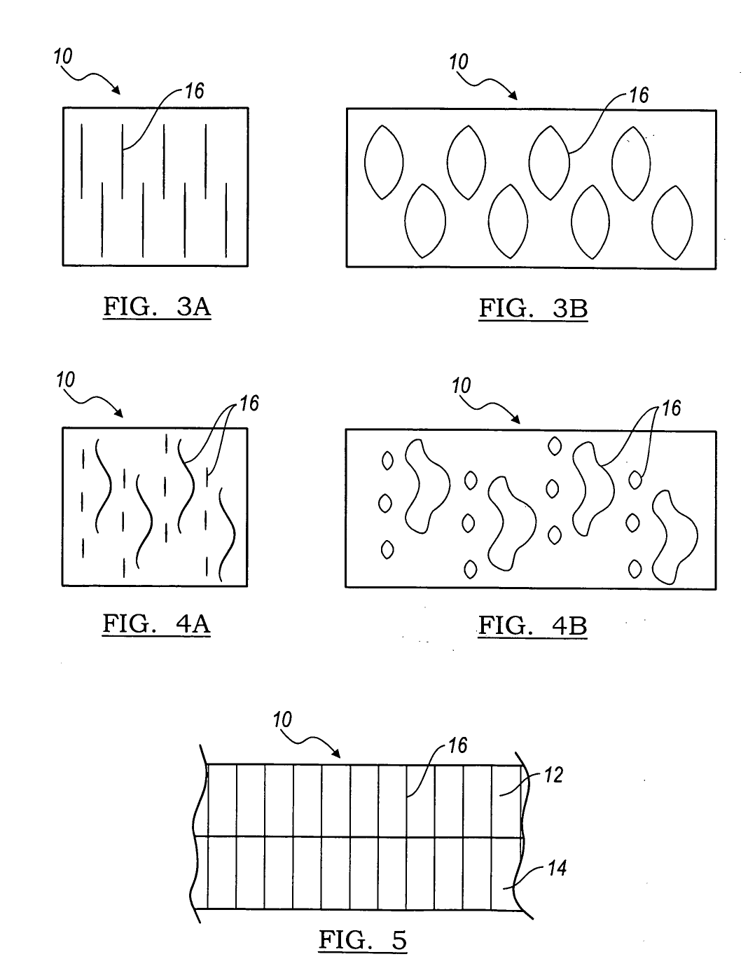

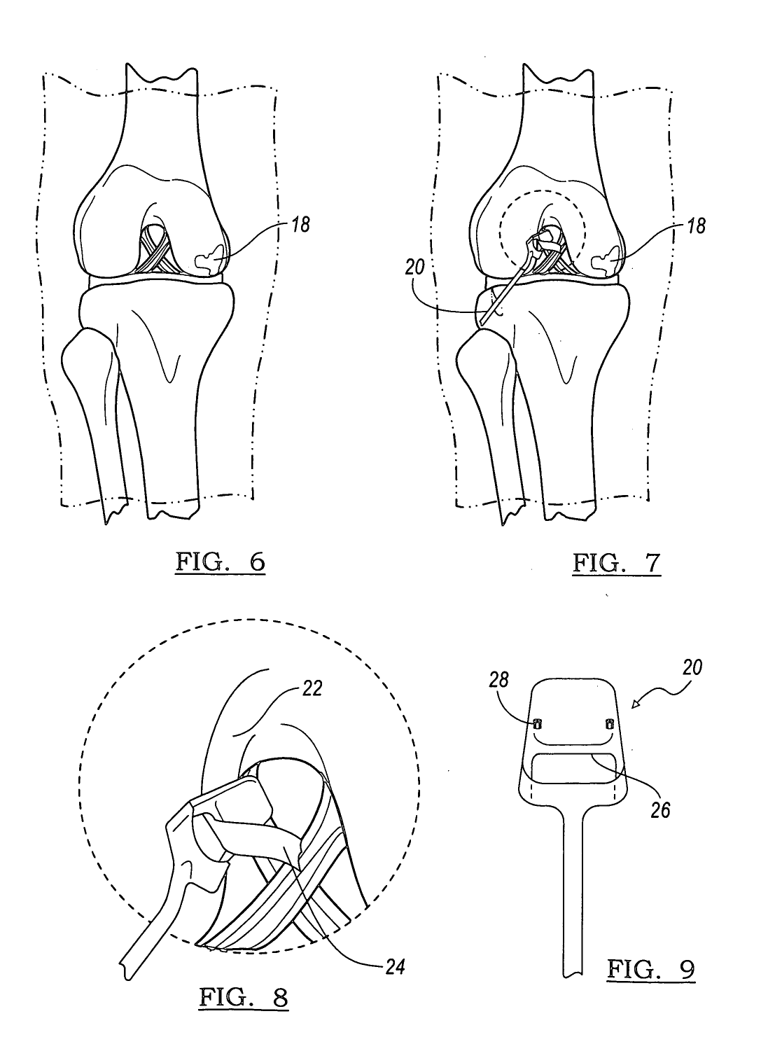

Referring to FIGS. 3A-3B, the whole superficial cartilage graft 10 can include at least one slit 16. In various embodiments, the whole superficial cartilage graft 10 includes a plurality of slits 16. The slits 16 generally have a first or unexpanded dimension. When the slits 16 are engaged, they open to their expanded dimension, which is greater than the unexpanded dimension, and thereby increase the surface area of the whole superficial cartilage graft 10. The slits 16 can have a first cross section or dimension in the unexpanded state. It is understood that when engaged, for example, when tension is placed on the cartilage graft 10, the slits 16 become larger, which in turns makes the cartilage graft 10 larger. The slits 16 can be equally distributed or randomly distributed across the whole superficial cartilage graft 10.

As depicted in FIG. 3A, a generally rectangular whole superficial cartilage graft 10 is provided. The whole superficial cartilage graft 10 includes longitudinal slits 16 which are parallel to the vertical sides of the rectangle. When the whole superficial cartilage graft 10 is engaged at the sides and pulled normal to the slits 16, either manually by gripping the edges of the whole superficial cartilage graft 10 in the hands and stretching the graft or using mechanical means such as a tool to grasp and secure one side of the graft 10 while the other side is stretched, the slits 16 expand and thereby increase the total surface area of the whole superficial cartilage graft 10, as shown in FIG. 3B.

The slits 16 can be of the same size within a single whole superficial cartilage graft 10 or a variety of slits 16 can be employed on a single whole superficial cartilage graft 10. Referring to FIGS. 4A and 4B, the whole superficial cartilage graft 10 includes free form double s-shaped slits 16. The sizes and shapes of the slits 16 allow the user to select the extent of expansion. Where an increased level of expansion is desired, more slits 16 can be incorporated into the whole superficial cartilage graft 10. In such embodiments where several slits 16 are used, the direction in which the partial thickness cartilage is expanded impacts the expansion amount. For example, as depicted in FIG. 4B, the whole superficial cartilage graft 10 is expanded horizontally, thereby causing an increase in the dimensions of the double s-shaped slits. If that whole superficial cartilage graft 10 was expanded vertically, there would be less expansion of the double s-shaped slits which may be desirable in cases where only minimal expansion is desired.

Referring to FIG. 5, the slits 16 can extend through the whole superficial cartilage graft 10. As depicted, the expandable surface feature 16 transverses both sides of the cartilage graft or extends through a top surface of the cartilage graft 10 to the bottom surface of the cartilage graft 10. Various sized slits 16 can also be selected for various regions of the whole superficial cartilage graft to provide enhanced strength where the graft 10 may be secured to an underlying tissue. The slits 16 are generally in the whole superficial cartilage graft 10 to allow for expansion of the graft, without compromising the monolithic structure of the whole superficial cartilage graft 10. In addition to allowing expansion of the whole superficial cartilage graft 10, the slits are also useful when the whole superficial cartilage graft 10 is placed over an arcuate surface and allow for more generous flexing of the whole superficial cartilage graft 10.

The shape and dimension of the slits 16 determine the expandability ratio of the whole superficial cartilage graft. In various embodiments, the expandability ratio ranges from about 1:1 up to about 1:10. The expandability ratio allows for a smaller sized whole superficial cartilage graft 10 to be placed in a larger defect site. This provides flexibility in the placement of the donor cartilage and also allows for a smaller graft to be used on a single larger defect. In various embodiments, it may be desirable to expand the whole superficial cartilage graft 10 at an amount below the maximum expansion ratio.

The whole superficial cartilage graft 10 can be coated with various agents to increase the ingrowth of new cartilage into the implant, facilitate securing the whole superficial cartilage graft 10 in an implant site, or promote tissue health. Exemplary agents include chondrocytes, undifferentiated cells, differentiation media, growth factors, platelet concentrate, nutrients, and combinations thereof. The agents can be selected for various reasons. For example, a differentiation media can be employed to enhance the chondrogenesis and promote differentiation of any undifferentiated cells which can be additionally applied to the area.

To obtain the whole superficial cartilage graft 10, a donor region 22 from which the cartilage will be excised is selected. In selecting a donor region 22 in a living being, it may be desirable to remove the cartilage from a non-load bearing region of the body. The cartilage can also be donated from a cadaveric source. The donor tissue can be from an allogeneic source or it can be from an autologous donation.

As depicted in FIGS. 7, 8, and 9, a slicing instrument 20 is used to remove the cartilage 24 from a non-load bearing donor region 22. The slicing instrument 20 includes a cutting edge or blade 26 to remove a predetermined thickness of cartilage. The blade 26 is adjustable to provide different cartilage thicknesses using the adjustment features 28. The cutting depth can be increased or decreased based on the dimension of the defect site 18. The blade 26, or any other appropriate cutting device, can be used to provide a whole superficial cartilage graft 10 having the superficial zone 12 and the intermediate zone 14 in the desired ratios. The blade 26, or any other appropriate cutting device, can also be used to shape the whole superficial cartilage graft 10 to the desired shape prior to implantation.

The thickness of the cartilage generally includes a thickness of the superficial zone 12 and the intermediate zone 14. The slicing instrument 20 is generally grasped by the user and pulled along the surface of the donor region 22 with sufficient pressure (which translates to cutting force of the blade 26) to provide an excised region of cartilage 24. It may be desirable to use the slicing instrument 20 such that the excised region of cartilage 24 can be easily manipulated or shaped into the whole superficial cartilage graft 10. This results in the excised region of cartilage 24 being used as the whole superficial cartilage graft 10 without any further trimming or manipulation.

In various embodiments, the whole superficial cartilage graft 10 can be meshed. Meshing as used herein, refers to the process of adding slits 16 or any other suitable expandable surface features, to increase the surface area coverage potential of the whole superficial cartilage graft 10. The meshing can be conducted by slicing, slitting, piercing, or employing a mechanical meshing apparatus.

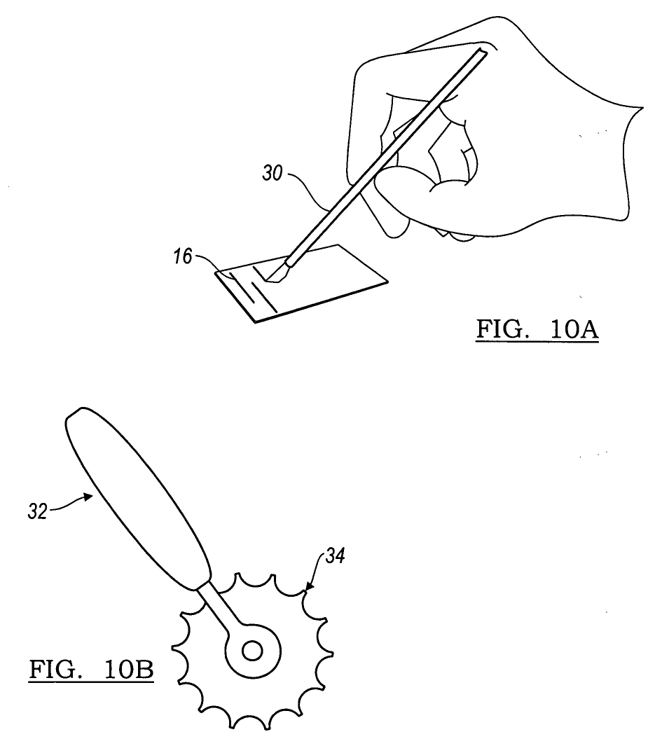

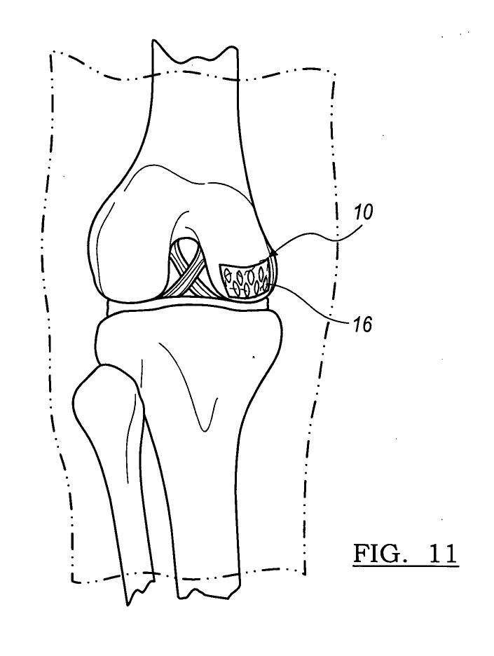

Referring to FIGS. 10A and 10B, various meshing instruments are provided. As shown in FIG. 10, the meshing instrument can be a scalpel 30. When using the scalpel 30, the user would cut slits 16 into the whole superficial cartilage graft 10. The slits 16 can be straight (FIG. 3A) or the slits can be free form (FIG. 4A). As shown in FIG. 10, a rolling device 32 having needles or blades 34 to cut slits 16 can be rolled over the whole superficial cartilage graft 10 to provide slits 16 for expansion of the whole superficial cartilage graft 10.

The defect site 18 can be prepared by removing at least a portion of the damaged tissue and optionally removing a small region of the healthy cartilage tissue to accommodate placement of the whole superficial cartilage graft 10. Exemplary defect sites 18 include, but are not limited to, a patella, a femoral condyle, a femoral head, and an acetabulum. Exemplary articular cartilage defects include those caused by trauma, excessive use (such as sports injuries, for example) or diseases, including, but not limited to, osteoarthritis and osteochondrosis dissecans.

Referring to FIG. 11, the whole superficial cartilage graft 10 is applied to the defect site 18. In embodiments where the whole superficial cartilage graft 10 includes expandable surface features 16, the whole superficial cartilage graft 10 can be expanded by stretching the graft or pulling the graft in a direction to cause the expandable surface features 16 and thereby increase the surface area of the whole superficial cartilage graft 10. In various embodiments, more than one whole superficial cartilage graft 10 is applied to the defect site. The multiple whole superficial cartilage grafts 10 can be fit together to cover regions of the defect site.

In various embodiments, the depth of tissue removal at the defect site 18 is of a thickness such that when inserted and secured, the whole superficial cartilage graft 10 can be flush with the surrounding tissue. A flush whole superficial cartilage graft 10 facilitates articulation in the region. In various embodiments, the whole superficial cartilage graft can be thicker than or slightly protrude above the plane of the surrounding tissue (for example, less than about 10% above the plane of the surrounding tissue). The whole superficial cartilage graft 10 can also be recessed within the defect site 18, as further detailed later herein.

Either in expanded or unexpanded form, the whole superficial cartilage graft 10 can be secured to the defect site 18 using any medically suitable securing device or technique, including, mechanical means, adhesive means, and combinations thereof. The securing method or methods used sufficiently reduce movement of the whole superficial cartilage graft 10. The whole superficial cartilage graft 10 will remain at the defect site and will not become unintentionally dislodged. The whole superficial cartilage graft 10 becomes an integral part of the defect site and normal daily use of the repaired area will not impact or move the whole superficial cartilage graft 10.

Mechanical attachment means include, but are not limited to, sutures, staples, tacks, and the like. Adhesive attachment means include, but are not limited to fibrin glue or other bioadhesives or sealants. In various embodiments, a membrane can be used to secure the whole superficial cartilage graft 10 in a defect site.

As a specific example, in those embodiments where the whole superficial cartilage graft 10 is recessed in the defect site 18 (for example, the whole superficial cartilage graft 10 is not planar with the highest region of the defect site), it may be desirable to use a membrane to cover the whole superficial cartilage graft 10. The membrane can include, but is not limited to, periosteal flap or hydrogel chitosan. Including the membrane over the recessed whole superficial cartilage graft 10 will retain the whole superficial cartilage graft 10 in the defect site 18 and serve as a protective barrier to prevent any unintentional dislodging of the whole superficial cartilage graft 10 from the defect site 18. The space between the membrane and the whole superficial cartilage graft 10 can be filled in or leveled with a fluid, such as sterile water or saline, for example. The membrane can be attached to the defect site using an additional securing method. For example, the periosteal flap can be sutured to the defect site 10 and then a fibrin sealant can be applied over the sutures.

Pre- or post-implantation preparation can also be utilized. The whole superficial cartilage graft and/or the defect site 18 can be coated with agents selected from chondrocytes, undifferentiated cells, differentiation media, growth factors, platelet concentrate, nutrients, and combinations thereof. The coating can be achieved by spreading or applying the agents across selected regions or the entire surface of the whole superficial cartilage graft 10 and/or the defect site 18. The coating can occur either before or after implanting the whole superficial cartilage graft 10. For example, in an embodiment where platelet concentrate is employed, the platelet concentrate can provide localized wound healing benefits to heal the wounds caused by surgical intervention.

The whole superficial cartilage graft 10 of the present teachings provides a simple and effective means of articular cartilage repair which is advantageous over the extensive preparation required for a full thickness cartilage graft. The preparation for the whole superficial cartilage graft 10 does not require removal of underlying subchondral bone. This may be advantageous where the underlying subchondral bone is still healthy. The superficial zone 12 provided to the defect site 18 provides the necessary chondrocytes for cartilage production. Also, the superficial zone 12 stimulates production of chondrocytes in the surrounding healthy tissue, thereby stimulating cartilage production and increasing the permanency of the whole superficial cartilage graft 10.

The description of the present teachings is merely exemplary in nature and, thus, variations that do not depart from the gist of the present teachings are intended to be within the scope of the present teachings. Such variations are not to be regarded as a departure from the spirit and scope of the present teachings.

What is claimed is:

1. A method for grafting whole superficial articular cartilage, comprising:
preparing a defect site to receive a whole superficial cartilage graft by removing a region of tissue;
obtaining only a whole superficial cartilage from a selected articular cartilage site; and
attaching the whole superficial cartilage to the prepared defect site to sufficiently reduce movement of the whole superficial cartilage.

2. The method of claim 1, further comprising preparing the whole superficial cartilage graft for the defect site by shaping the whole superficial cartilage graft to correspond with at least a region of the defect site.

3. The method of claim 1, further comprising treating at least one of the defect site or the whole superficial cartilage graft with an agent to enhance growth of the whole superficial cartilage graft.

4. The method of claim 1, wherein attaching the whole superficial cartilage graft to the defect site is performed using a securing method selected from the group consisting of: mechanical securing, adhesive securing, membrane securing, and combinations thereof.

5. The method of claim 1, wherein obtaining the whole superficial cartilage graft comprises harvesting the whole superficial cartilage graft from an allogeneic donor and preserving the graft for later use.

6. The method of claim 1, wherein obtaining the whole superficial cartilage graft comprises harvesting the whole superficial cartilage graft from an autologous articular cartilage.

7. The method of claim 1, wherein the whole superficial cartilage graft further includes an integral layer of intermediate cartilage.

8. The method of claim 1, further comprising forming at least one slit in the whole superficial cartilage graft to allow the graft to expand under tension to fill at least a region of the defect.

9. The method of claim 8, further comprising forming a plurality of slits; wherein the slits are equally distributed across the surface of the whole superficial cartilage graft.

10. The method of claim 8, wherein the whole superficial cartilage graft has an expansion ratio of from about 1:1 to about 1:10.

11. The method of claim 8, further comprising tensioning the whole superficial cartilage graft to expand the slit and thereby expand the superficial cartilage graft.

12. The method of claim 1, wherein preparing the defect site is performed on a body region selected from the group consisting of: a patella, a femoral condyle, a femoral head, and an acetabulum.

13. The method of claim 1, further comprising coating at least a region of the whole superficial cartilage graft with an agent to promote cartilage growth; wherein the agent is selected from the group consisting of: chondrocytes, undifferentiated cells, differentiation media, growth factors, platelet concentrate, nutrients, and combinations thereof.

14. A method for repairing knee articular cartilage defects, comprising:
preparing a defect site located on a condyle surface by removing a region of tissue;
harvesting only a whole autologous superficial cartilage from a selected articular cartilage site;
applying the whole autologous superficial cartilage to the prepared defect site; and
attaching the whole autologous superficial cartilage to the prepared defect site to sufficiently reduce movement of the whole autologous superficial cartilage.

15. The method of claim 14, further comprising preparing the whole autologous superficial cartilage graft by sizing the whole autologous superficial cartilage graft to fit the defect site.

16. The method of claim 14, wherein the whole autologous superficial cartilage includes an integral layer of intermediate cartilage.

17. The method of claim 14, wherein attaching the whole autologous superficial cartilage graft to the defect site is selected from a securing method selected from the group consisting of: mechanical securing, adhesive securing, membrane securing, and combinations thereof.

18. A method for grafting whole superficial articular cartilage, comprising:
preparing a defect site to receive a whole superficial cartilage graft by removing a region of tissue;
obtaining only whole superficial cartilage from a selected articular cartilage site;
placing the whole superficial cartilage in the defect site;
covering the whole superficial cartilage graft in the defect site with a periosteal flap; and
attaching the periosteal flap to the prepared defect site.

19. The method of claim 18, further comprising applying a fibrin sealant over at least a region of the periosteal flap.

20. The method of claim 18, further comprising leveling the attached periosteal flap defect site with at least a region of a surrounding healthy tissue.
